(19) United States
(12) Patent Application Publication
(10) Pub. No.: US 2015/0093439 A1
(43) Pub. Date: Apr. 2, 2015

(54) COMPRESSION COATED PULSATILE RELEASE COMPOSITIONS

(71) Applicant: McNeil-PPC, Inc., Skillman, NJ (US)

(72) Inventors: Der-Yang Lee, Flemington, NJ (US); Shun-Por Li, Landsdale, PA (US); Timothy Kutch, Paoli, PA (US)

(21) Appl. No.: 14/498,698

(22) Filed: Sep. 26, 2014

Related U.S. Application Data
(60) Provisional application No. 61/883,590, filed on Sep. 27, 2013.
(60) Provisional application No. 62/030,310, filed on Jul. 29, 2014.

(51) Int. Cl. A61K 31/192; A61K 9/20; A61K 9/28

(52) U.S. Cl. CPC A61K 31/192; A61K 9/2866; A61K 9/2095; A61K 9/2893
USPC 424/480; 514/570

(57) ABSTRACT

The present invention is directed to a dosage form comprising an immediate release portion of a first active pharmaceutical ingredient and a delayed release portion of a second active pharmaceutical ingredient wherein (a) the immediate release portion comprises from about 1 mg to about 1000 mg of the first active pharmaceutical ingredient; and (b) the delayed release portion comprises from about 1 mg to about 1000 mg of the second active pharmaceutical ingredient; wherein the delayed release portion is coated with a delayed release coating comprising at least one swellable erodible polymer and a filler, and wherein the immediate release portion is in contact with the delayed release coating.

### CROSS-REFERENCE TO RELATED APPLICATIONS

This application claims priority of the benefits of the filing of U.S. Provisional Application Ser. No. 61/883,590, filed Sep. 27, 2013 and U.S. Provisional Application Ser. No. 62/030,310, filed Jul. 29, 2014, the contents of each of which are hereby incorporated by reference in their entirety.

### BACKGROUND OF THE INVENTION

1. Field of the Invention

The present invention relates to a solid dosage form containing an active pharmaceutical ingredient, wherein the active pharmaceutical ingredient is delivered in a pulsatile manner. The method of making the solid dosage form is also provided.

2. Background of the Invention

Therapeutic agents for treating pain, inflammation, and fever include analgesics, anti-inflammatories, and antipyretics. Non-steroidal anti-inflammatory drugs (NSAID's) are one type of such therapeutic agents. They include propionic acid derivatives, acetic acid derivatives, fenamic acid derivatives, biphenylcarbodylic acid derivatives, oxicams, and cyclooxygenase-2 (COX-2) selective NSAID's.

Propionic acids include for example ibuprofen, naproxen, and ketoprofen. Ibuprofen in particular is a widely used, well known NSAID possessing analgesic and antipyrretic properties. It has been commercially available as an over-the-counter drug in many forms for several years. Ibuprofen is chemically known as 2-(4-isobutylphenyl)-propionic acid.

NSAID's are typically administered on a once to four times daily basis, with the daily dose ranging from about 50 to about 2000 milligrams, preferably from about 100 to 1600 and most preferably from about 200 to about 1200 milligrams.

Acetaminophen (APAP) is a well-known analgesic, with a daily dose ranging from about 325 to about 4000 milligrams, preferably from about 650 to about 4000 milligrams. Considering the widespread use of APAP and the volume of its manufacture, both its manufacture and its use as an analgesic are well known to persons skilled in the art.

It is known to administer NSAID's, acetaminophen, and other drugs in multiple doses over 12 or 24 hours. For example, it is known to administer multiple doses containing equal amounts of ibuprofen over 12 to 24 hours. Sustained release dosage forms containing ibuprofen are also known.

It is useful to minimize the “drug exposure” of a patient. In other words, to administer the least total amount of drug that will provide the optimal beneficial therapeutic effect. In particular, it is useful to administer analgesics such as NSAIDs or acetaminophen in a regimen which provides maximal relief at minimal total dose per day of drug.

Applicants have now created a dosage form, which can provide a two step dosing regimen with improved therapeutic effect, especially pain relief.

### SUMMARY OF THE INVENTION

The present invention is directed to a dosage form comprising an immediate release portion of a first active pharmaceutical ingredient and a delayed release portion of a second active pharmaceutical ingredient wherein (a) the immediate release portion comprises from about 1 mg to about 1000 mg of the first active pharmaceutical ingredient; and (b) the delayed release portion comprises from about 1 mg to about 1000 mg of the second active pharmaceutical ingredient; wherein the delayed release portion is coated with a delayed release coating comprising at least one swellable erodible polymer and a filler, and wherein the immediate release portion is in contact with the delayed release coating.

The present invention also includes a process for the manufacture of an immediate release portion of a first active pharmaceutical ingredient and a delayed release portion of a second active pharmaceutical ingredient, the method comprising (a) obtaining a core comprising from about 1 mg to about 1000 mg of a first active pharmaceutical ingredient; (b) compressing a powder on the surface of the core to form a delayed release coating on the surface of the core, wherein the powder comprises at least one swellable erodible polymer and a filler; and (c) compressing a second powder onto the surface of the delayed release coating, wherein the second powder comprises from about 1 mg to about 1000 mg of a first active pharmaceutical ingredient; wherein the immediate release portion comprises the compressed second powder, and the delayed release portion comprises the core and the delayed release coating.

### BRIEF DESCRIPTION OF THE DRAWINGS

The detailed description will be better understood in conjunction with the accompanying drawings:

FIG. 1 depicts a dissolution graph of the tablets described in Example 2(d);

FIG. 2 depicts a dissolution graph of the tablets described in Example 2(e);

FIG. 3 depicts a dissolution graph of the tablets described in Example 2(f);

FIG. 4 depicts a dissolution graph of the tablets made in Example 8 using Compression Method A (from Example 8);

FIG. 5 depicts a dissolution graph of the tablets made in Example 9, Part A (10% wax) using Compression Method B (from Example 8) at a compression coating of 1.8× core weight;

FIG. 6 depicts a dissolution graph of the tablets made in Example 9, Part A (10% Wax) using Compression Method A (from Example 8) at a compression coating of 2× core weight, and Example 9, Part A (10% Wax) using Compression Method B (from Example 8) at a compression coating of 2× core weight;

FIG. 7 depicts a dissolution graph of the tablets made in Example 9, Part A (10% Wax) using Compression Method B (from Example 8) at a compression coating of 2.3× core weight;

FIG. 8 depicts a dissolution graph of the tablets made in Example 9, Part A (10% Wax) using Compression Method A (from Example 8) at a compression coating of 2.6× core weight;

FIG. 9 depicts a dissolution graph of the tablets made in Example 9, Part B (30% Wax, 15% HPMC K4MCR, 30% HPC EXF) using Compression Method B (from Example 8) at a compression coating of 2.6× core weight;

FIG. 10 depicts a dissolution graph of the tablets made in Example 9, Part C (30% Wax, 13% HPMC K4MCR, 26% HPC EXF) using Compression Method B (from Example 8) at a compression coating of 2.6× core weight;

FIG. 11 depicts a dissolution graph of the tablets made in Example 9, Part D (60% Wax) using Compression Method B (from Example 8) at a compression coating of 2.6× core weight; and

FIG. 12 depicts a dissolution graph of the tablets made in Example 10, at a compression coating of 2.6× core weight.

### DETAILED DESCRIPTION OF THE INVENTION

The present invention includes a composition for compression coating a core of an active pharmaceutical ingredient (“API”), in order to achieve a tablet dosage form with a pH independent pulsatile (pulse) release. The pulse release is intended to provide a delayed release dose of an API about 2 to about 8 hours, preferably about 2 to about 6 hours, more preferably about 3 to about 6 hours, and even more preferably about 4 to about 6 hours after the initial start of dosing.

In another embodiment, the present invention is directed to a dosage form comprising both an immediate release portion of a first active pharmaceutical ingredient and a delayed release portion of a second active pharmaceutical ingredient. The immediate release portion comprises from about 1 mg to about 1000 mg of the first active pharmaceutical ingredient and the delayed release portion comprises from about 1 mg to about 1000 mg of the second active pharmaceutical ingredient. The delayed release portion is coated with a delayed release coating comprising at least one swellable erodible polymer and the immediate release portion is in contact with the delayed release coating.

The solid dosage form is designed to deliver from about 1 mg to about 1000 mg of active pharmaceutical ingredient in the immediate release portion. Preferably, the immediate release portion delivers about 1 mg to about 500 mg, and more preferably, about 1 mg to about 400 mg of the solid dosage form. Even more preferably, the immediate release portion delivers about 100 mg to about 400 mg, and still even more preferably, about 150 mg to about 400 mg of the solid dosage form. The dosage form contains a specific level of active pharmaceutical ingredient depending on whether one or two tablets are ingested at a specific dosing time. Two tablets may be preferable for ease of dosing and swallowing. In one embodiment, where one tablet is ingested, the immediate release portion contains about 300 mg to about 400 mg active pharmaceutical ingredient. In another embodiment, where two tablets are ingested at a single time, the immediate release portion contains about 150 to about 200 mg active pharmaceutical ingredient per tablet.

The delayed release portion of the solid dosage form is designed to include from about 1 mg to about 1000 mg of an active pharmaceutical ingredient. Preferably, the active pharmaceutical ingredient is about 1 mg to about 500 mg, and more preferably, about 1 mg to about 400 mg. Even more preferably, the active pharmaceutical ingredient is about 100 mg to about 400 mg, and still even more preferably, about 150 mg to about 400 mg. In one embodiment, where one tablet is ingested, the delayed release portion contains about 100 mg to about 300 mg active pharmaceutical ingredient. In another embodiment, where two tablets are ingested at a single time, the delayed release portion contains about 100 to about 150 mg active pharmaceutical ingredient per tablet.

The first API and second API may be any active pharmaceutical ingredient. For example, analgesics, anti-inflammatories, antipyretics, antihistamines, decongestants, cough suppressants and expectorants, muscle relaxants, stimulants, sedatives, appetite suppressants, anesthetics, statins, and the like.

The dosage form of the present invention includes a first active ingredient and a second active ingredient. The first and second active ingredients may be, for example, acetaminophen, aspirin, naproxen, ketoprofen, flurbiprofen, diclofenac, cyclobenzaprine, meloxicam, rofecoxib, celecoxib, and pharmaceutically acceptable salts, esters, isomers, and mixtures thereof and combinations thereof. Other Suitable active ingredients for use in this invention include analgesics, anti-inflammatory agents, antiarthritics, anesthetics, antihistamines, antitussives, antibiotics, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, decongestants, oral contraceptives, diuretics, expectorants, gastrointestinal agents, migraine preparations, motion sickness products, mucolytics, muscle relaxants, osteoporosis preparations, polydimethylsiloxanes, respiratory agents, sleep-aids, urinary tract agents and mixtures thereof.

Examples of suitable analgesics, anti-inflammatories, and antipyretics include, but are not limited to, non-steroidal anti-inflammatory drugs (NSAIDs) such as propionic acid derivatives (e.g., sodium ibuprofen, ibuprofen, naproxen, ketoprofen, flurbiprofen, fenbufen, fenoprofen, indoprofen, ketoprofen, fluprofen, pirprofen, carprofen, oxaprozin, pranoprofen, and suprofen) and COX inhibitors such as celecoxib; acetaminophen; acetyl salicylic acid; acetic acid derivatives such as indomethacin, diclofenac, sulindac, and tolmetin; fenamic acid derivatives such as mefanamic acid, meclofenamic acid, and flufenamic acid; biphenylcarbodylic acid derivatives such as diflunisal and flufenisal; and oxicams such as piroxicam, sudoxicam, isoxicam, and meloxicam; isomers thereof; and pharmaceutically acceptable salts and prodrugs thereof.

Examples of antihistamines and decongestants, include, but are not limited to, bromopheniramine, chlorcyclizine, dexbrompheniramine, bromhexane, phenindamine, pheniramine, pyrilamine, thonzylamine, pripolidine, ephedrine, phenylephrine, pseudoephedrine, phenylpropanolamine, chlorpheniramine, dextromethorphan, diphenhydramine, doxylamine, astemizole, terfenadine, fexofenadine, naphazoline, oxymetazoline, montelukast, propylhexadrine, triprolidine, clemastine, acrivastine, promethazine, oxomemazine, mequitazine, buclizine, bromhexine, ketotifen, terfenadine, ebastine, oxatamide, xylomeazoline, loratadine, desloratadine, and cetirizine; isomers thereof; and pharmaceutically acceptable salts and esters thereof.

Examples of cough suppressants and expectorants include, but are not limited to, diphenhydramine, dextromethorphan, noscapine, clophedianol, menthol, benzonatate, ethylmorphone, codeine, acetylcysteine, carbocisteine, ambroxol, belladona alkaloids, sobrenol, guaiacol, and guaifenesin; isomers thereof; and pharmaceutically acceptable salts and prodrugs thereof.

Examples of muscle relaxants include, but are not limited to, cyclobenzaprine and chlorzoxazone metaxalone, orphenadrine, and methocarbamol; isomers thereof; and pharmaceutically acceptable salts and prodrugs thereof.

Examples of stimulants include, but are not limited to, caffeine.

Examples of sedatives include, but are not limited to sleep aids such as antihistamines (e.g., diphenhydramine), eszopiclone, and zolpidem, and pharmaceutically acceptable salts and prodrugs thereof.

Examples of appetite suppressants include, but are not limited to, phenylpropanolamine, phentermine, and diethylcathinone, and pharmaceutically acceptable salts and prodrugs thereof.

Examples of anesthetics (e.g., for the treatment of sore throat) include, but are not limited to dyclonine, benzocaine, and pectin and pharmaceutically acceptable salts and prodrugs thereof.

Examples of suitable statins include but are not limited to atorvastin, rosuvastatin, fluvastatin, lovastatin, simvustatin, atorvastatin, pravastatin and pharmaceutically acceptable salts and prodrugs thereof.

Examples of suitable gastrointestinal agents include antacids such as calcium carbonate, magnesium hydroxide, magnesium oxide, magnesium carbonate, aluminum hydroxide, sodium bicarbonate, dihydroxyaluminum sodium carbonate; stimulant laxatives, such as bisacodyl, cascara sagrada, danthron, senna, phenolphthalein, aloe, castor oil, ricinoleic acid, and dehydrocholic acid, and mixtures thereof; H2 receptor antagonists, such as famotidine, ranitidine, cimetadine, nizatidine; proton pump inhibitors such as omeprazole or lansoprazole; gastrointestinal cytoprotectives, such as sucraflate and misoprostol; gastrointestinal prokinetics, such as prucalopride, antibiotics for H. pylori, such as clarithromycin, amoxicillin, tetracycline, and metronidazole; antidiarrheals, such as diphenoxylate and loperamide; glycopyrrolate; antiemetics, such as ondansetron, analgesics, such as mesalamine.

In one embodiment of the invention, the first and/or second active ingredient may be selected from pseudoephedrine, pheylephrine, phenylpropanolamine, chlorpheniramine, dextromethorphan, diphenhydramine, astemizole, terfenadine, fexofenadine, loratadine, desloratadine, cetirizine, mixtures thereof and pharmaceutically acceptable salts, esters, isomers, acetaminophen, nicotine, ranitidine, ibuprofen, ketoprofen, loperamide, famotidine, calcium carbonate, simethicone, methocarbomal, chlophedianol, ascorbic acid, pectin, dyclonine, benzocaine and menthol, their pharmaceutically acceptable salts and prodrugs thereof, and mixtures thereof.

In another embodiment, the immediate release portion comprises ibuprofen and derivatives thereof. In yet another embodiment, the immediate release portion comprises ibuprofen sodium and the delayed release portion comprises ibuprofen. In still yet another embodiment, the immediate release portion comprises ibuprofen sodium and the delayed release portion comprises ibuprofen sodium.

The first API and second API may be the same or different.

In one embodiment, the amount of the first API to the amount of the second API is about 1:1 to about 2:1.

In one embodiment, the API has a solubility greater than about 1 mg/ml. In another embodiment, the API has a solubility greater than about 100 mg/ml. In yet another embodiment, the API has a solubility greater than about 150 mg/ml. In still yet another embodiment, the API has a solubility greater than about 200 mg/ml. In even still yet another embodiment, the API has a solubility greater than about 250 mg/ml. It should be noted that the solubility is determined at 25° C.

The coating of the current invention includes at least one swellable edible polymer. Suitable swellable edible polymers include, but are not limited to, hydroxypropylcellulose (HPC, Klucel® HF), hypromelllose (HPMC K15M or HPMC K4M), hydroxyethylcellulose, and mixtures thereof. Additional swellable edible polymers include, for example, hydroxypropyl cellulose, hydroxypropyl methylcellulose (Methocel K100M), hydroxyethylcellulose and mixtures thereof. In one embodiment, the high molecular weight, water soluble polymer comprises hydroxypropyl cellulose having a weight average molecular weight from about 80,000 to about 1,150,000. In another embodiment, the high molecular weight, water soluble polymer comprises hydroxypropyl methylcellulose (e.g., hypromellose) having a viscosity from about 75 to about 120,000 cps in 2% aqueous solution at 20° C.

Still further examples of swellable erodible hydrophilic materials for use as release-modifying excipients for making the coating, or a portion thereof, include: water swellable cellulose derivatives, polyalkalene glycols, thermoplastic polyalkalene oxides, acrylic polymers, hydrocolloids, clays, gelling starches, and swelling cross-linked polymers, and derivatives, copolymers, and combinations thereof. Examples of suitable swellable erodible cellulose derivatives include sodium carboxymethylcellulose, cross-linked hydroxypropylcellulose, hydroxypropyl cellulose (HPC), hydroxypropylmethylcellulose (HPMC), hydroxyisopropylcellulose, hydroxybutylcellulose, hydroxyphenylcellulose, hydroxyethylcellulose (HEC), hydroxypentylcellulose, hydroxypropylethylcellulose, hydroxypropylbutylcellulose, hydroxypropylethylcellulose. Examples of suitable polyalkalene glyclols include polyethylene glycol. Examples of suitable thermoplastic polyalkalene oxides include poly(ethylene oxide). Examples of suitable acrylic polymers include potassium methacrylatedivinylbenzene copolymer, polymethylmethacrylate, CARBOPOL (high-molceular weight cross-linked acrylic acid homopolymers and copolymers), and the like. Examples of suitable hydrocolloids include alginates, agar, guar gum, locust bean gum, kappa carrageenan, iota carrageenan, tara, gum arabic, tragacanth, pectin, xanthan gum, gellan gum, maltodextrin, galactomannan, pusstulan, laminarin, scleroglucan, gum arabic, inulin, pectin, gelatin, whelan, rhamsan, zooglan, methylan, chitin, cyclodextrin, chitosan.

The amount of swellable edible polymer included in the coating is from about 2 percent to about 60 percent by weight of the coating. Preferably, the swellable edible polymer is about 2 percent to about 50 percent, and more preferably, about 4 percent to about 40 percent by weight of the coating in the solid dosage form.

In some embodiments, the coating comprises two types of swellable edible polymers. The first swellable edible polymer may be present from about 1 percent to about 50 percent by weight of the coating, preferably from about 10 percent to about 40 percent by weight of the coating, more preferably from about 20 to about 35 percent by weight of the coating. The second swellable edible polymer may be present from about 1 percent to about 20 percent by weight of the coating, preferably from about 10 percent to about 20 percent by weight of the coating, more preferably from about 12 percent to about 18 percent by weight of the coating. In one embodiment the first swellable edible polymer is hydroxypropylcelloluse and the second swellable edible polymer is hypromellose. In another embodiment, the first swellable edible polymer is hypromellose and the second swellable edible polymer is hydroxypropylcelloluse.

In one embodiment, the amount of the first swellable edible polymer to the amount of the second swellable edible polymer is about 1:1 to about 2:1.

The dosage form also includes a filler. In one embodiment the coating comprises a filler from about 10 percent to about 99 percent by weight of the coating, preferably from about 30 percent to about 99 percent by weight of the coating, more preferably from about 40 percent to about 99 percent by weight of the coating, and even more preferably from about 40 percent to about 60 percent by weight of the coating. Non-limiting examples include, lactose, microcrystalline cellulose, and mixtures thereof.

Suitable fillers may include water soluble (e.g., carbohydrates) fillers such as, for example, lactose, dextrose, sucrose, mannose, mannitol, sorbitol, erythitol, xylitol, or mixtures thereof.

In one embodiment, the filler material may be a water insoluble material such as a low-melting hydrophobic material and/or a water insoluble polymer.

Suitable low-melting hydrophobic materials include water insoluble fillers such as, for example, fats, fatty acid esters, phospholipids, waxes, vegetable oils, free fatty acids and their salts, phospholipidsfats, fatty acid esters, phospholipids, and waxes.

Examples of suitable fats include hydrogenated vegetable oils such as for example cocoa butter, hydrogenated palm kernel oil, hydrogenated cottonseed oil, hydrogenated sunflower oil, and hydrogenated soybean oil; and free fatty acids and their salts. Examples of suitable fatty acid esters include sucrose fatty acid esters, mono, di, and triglycerides, glyceryl behenate, glyceryl palmitostearate, glyceryl monostearate, glyceryl tristearate, glyceryl trilaurylate, glyceryl myristate, GlycoWax-932, lauroyl macrogol-32 glycerides, and stearoyl macrogol-32 glycerides. Examples of suitable phospholipids include phosphotidyl choline, phosphotidyl serene, phosphotidyl enositol, and phosphotidic acid. Examples of suitable waxes include carnauba wax, spermaceti wax, beeswax, candelilla wax, shellac wax, microcrystalline wax, and paraffin wax; and the like.

The coating composition may also include water insoluble polymers. Examples of suitable water-insoluble polymers include ethylcellulose, polyvinyl alcohols, polyvinyl acetate, polycaprolactones, cellulose acetate and its derivatives, acrylates, methacrylates, acrylic acid copolymers; and the like and derivatives, copolymers, and combinations thereof.

In one embodiment, the coating composition comprises a water soluble filler and a water insoluble filler.

Optionally, other ingredients may be included in the composition or dosage form of the present invention.

Other ingredients or components may be added to the composition including but not limited to superdisintegrants, lubricants, glidants, aromas; sweeteners such as, sorbitol, sugar, and high intensity sweeteners such as sucralose, aspartame and saccharine and the like may be included.

Any coloring agent suitable for use in a food or pharmaceutical product may be used in the present inventive composition or dosage form. Typical coloring agents include, for example, azo dyes, quinopthalone dyes, triphenylmethane dyes, xanthene dyes, indigoid dyes, iron oxides, iron hydroxides, titanium dioxide, natural dyes, and mixtures thereof. More specifically, suitable colorants include, but are not limited to patent blue V, acid brilliant green BS, red 2G, azorubine, ponceau 4R, amaranth, D&C red 33, D&C red 22, D&C red 26, D&C red 28, D&C yellow 10, FD&C yellow 5, FD&C yellow 6, FD&C red 3, FD&C red 40, FD&C blue 1, FD&C blue 2, FD&C green 3, brilliant black BN, carbon black, iron oxide black, iron oxide red, iron oxide yellow, titanium dioxide, riboflavin, carotenes, antyhocyanines, turmeric, cochineal extract, clorophyllin, canthaxanthin, caramel, betanin, and mixtures thereof.

Similarly, a flavor may be included in the composition or solid dosage form. The amount of flavor added to the composition will be dependent upon the desired taste characteristics.

The purpose of the invention is to provide a dosage form with a lag time (delay) for the release of a portion of active ingredient, wherein a minimal amount of active ingredient is released for a determined amount of time (2-8 hours), followed by a burst release of active ingredient with a short period of time, for example, less than 60 minutes, preferably less than 30 minutes, following the lag time. In one embodiment, following a 6 hour lag time, the active ingredient may be present at less than 5 percent released for 6 hours, followed by greater than 80 percent released in 60 additional minutes, e.g., 7.0 hours. Various ratios of these materials would provide varying lag times. Additionally, the thickness of the coating can affect the release rate and/or lag time. A lubricant and glidant may be added to the compression coating blend in order to facilitate compression.

The dosage form of the present invention may be made by any method known to those skilled in the art so long as it results in the desired composition.

In one embodiment, the process used to manufacture an immediate release portion of a first active pharmaceutical ingredient and a delayed release portion of a second active pharmaceutical ingredient, comprises the steps of (a) obtaining a core comprising from about 1 mg to about 1000 mg of a first active pharmaceutical ingredient; (b) compressing a powder on the surface of the core to form a delayed release coating on the surface of the core, wherein the powder comprises at least one swellable erodible polymer and a filler; and (c) compressing a second powder onto the surface of the delayed release coating, wherein the second powder comprises from about 1 mg to about 1000 mg of a first active pharmaceutical ingredient; wherein the immediate release portion comprises the compressed second powder, and the delayed release portion comprises the core and the delayed release coating.

In another embodiment, the process used to manufacture an immediate release portion of a first active pharmaceutical ingredient and a delayed release portion of a second active pharmaceutical ingredient, the method comprising for the manufacture of a dosage form comprising an immediate release portion of a first active pharmaceutical ingredient and a delayed release portion of a second active pharmaceutical ingredient, the method comprising (a) obtaining a core comprising from about 1 mg to about 1000 mg of the first active pharmaceutical ingredient; (b) compressing a coating on the surface of the core to form a delayed release coating on the surface of the core, wherein the coating comprises at least one swellable erodible polymer and a filler; and (c) compressing an immediate release powder onto the surface of the delayed release coating, wherein the immediate release powder comprises from about 1 mg to about 1000 mg of a first active pharmaceutical ingredient; wherein the immediate release portion comprises the compressed immediate release powder and the delayed release portion comprises the core and the delayed release coating.

The following examples are provided to further illustrate the compositions and methods of the present invention. It should be understood that the present invention is not limited to the examples described.

Examples of compression coating blends are shown in the following TABLES. The core was compressed between 103 and 106 mg, and the coating level was approximately three times (3X) the core weight, with a whole tablet weight between 418 and 429 mg.

### Example 1

The core that was used included ibuprofen, sodium starch glycolate (EXPLOTAB), and fumed silica (Cab-O-Sil).

TABLE 1
Compression Coating Blend Formulations (as a % of the formulation)

| Material | Sample 1 | Sample 2 | Sample 3 | Sample 4 | Sample 5 | Sample 6 |
|---|---|---|---|---|---|---|
| Hydroxypropylcellulose | 10 | 10 | 10 | 10 | 20 | 30 |
| (Klucel HF) | | | | | | |
| Hydroxypropylcellulose | 10 | 15 | 15 | 15 | 25 | 25 |
| (Klucel EF) | | | | | | |
| Microcrystalline | 46 | 44 | | | | |
| Cellulose | | | | | | |
| (Avicel PH102) | | | | | | |
| Lactose (Fast Flow) | 33 | 30 | 50 | 75 | 55 | 45 |
| Fumed Silica (Cab-O- | 0.5 | 0.5 | | | | |
| Sil) | | | | | | |
| Mag Stearate | 0.5 | 0.5 | | | | |
| Dicalcium Phosphate | | | 25 | | | |
| Dihydrate (Di-tab) | | | | | | |

TABLE 2
Compression Coating Blend Formulations (as a % of the formulation)

| | Sample | Sample | Sample | Sample | Sample | Sample | Sample |
|---|---|---|---|---|---|---|---|
| Material | 7 | 8 | 9 | 10 | 11 | 12 | 13 |
| Hydroxypropyl | 20 | 10 | 30 | 15 | 15 | 15 | |
| methylcellulose (HPMC | | | | | | | |
| K15M) | | | | | | | |
| Hydroxypropylcellulose | 25 | 15 | 25 | | | | |
| (Klucel EF) | | | | | | | |
| Hydroxypropyl | | | | | | | 20 |
| methylcellulose (HPMC | | | | | | | |
| K4M) | | | | | | | |
| Hydroxypropylcellulose | | | | 15 | 20 | 25 | 25 |
| (Klucel EXAF) | | | | | | | |
| Lactose (Fast Flow) | 55 | 75 | 45 | 70 | 65 | 60 | 55 |

TABLE 3
Compression Coating Blend Formulations
(as a % of the formulation)

| | Sample | Sample | Sample | Sample |
|---|---|---|---|---|
| Material | 14 | 15 | 16 | 17 |
| Hydroxypropyl | 10 | 12.5 | 12.5 | |
| methylcellulose (HPMC | | | | |
| K15M) | | | | |
| Hydroxypropyl | | | | 15 |
| methylcellulose (HPMC | | | | |
| K4M) | | | | |
| Hydroxypropylcellulose | 15 | 12.5 | 15 | 30 |
| (Klucel EXAF) | | | | |
| Lactose (Fast Flow) | 75 | 75 | 77.5 | 55 |

### Example 2

The core that was used included ibuprofen, sodium starch glycolate (EXPLOTAB), and fumed silica (Cab-O-Sil).

### Part A: Pulse Release Coating Formula:

(a) The lactose, hydroxypropylmethylcellulose and hydroxypropylcellulose were de-lumped through a #40 mesh sieve.
(b) The delumped lactose, hydroxypropylmethylcellulose, hydroxypropylcellulose (a) were mixed in a suitable v-blender for 15 minutes at 25 RPM.

### Compression Coating Tabletting:

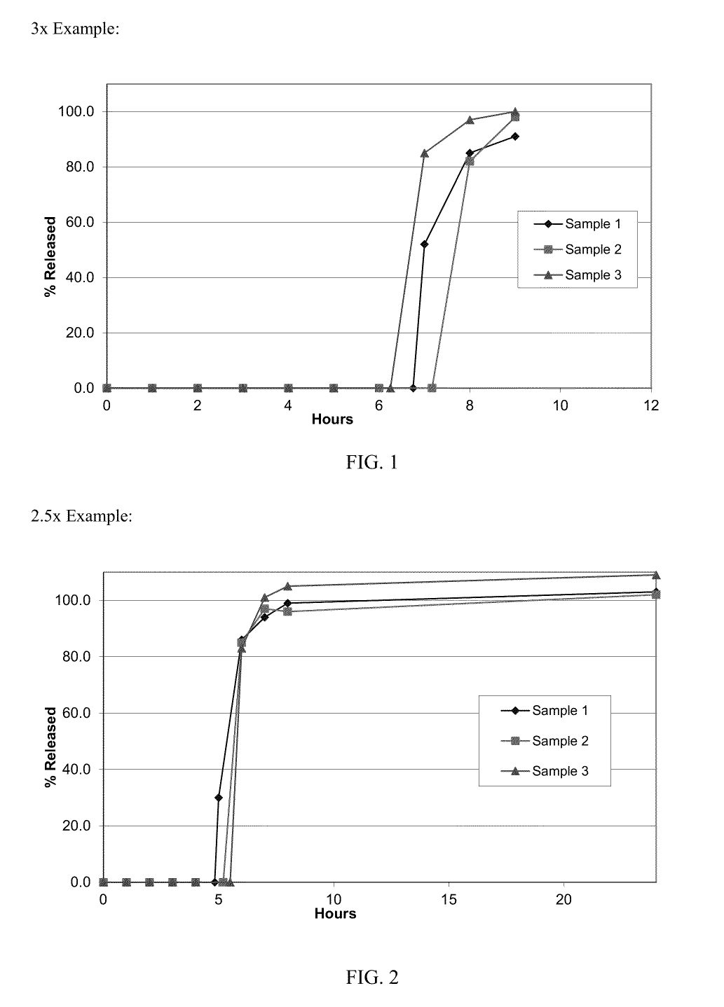

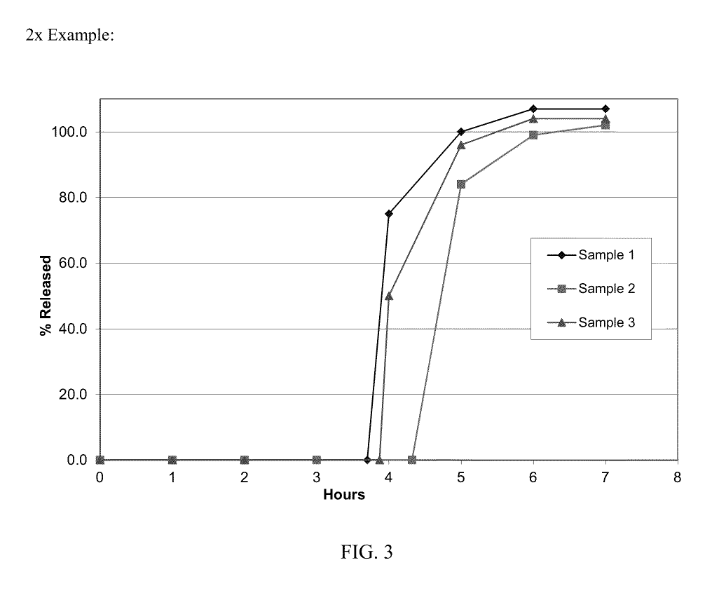

(a) The required amount of the blend from TABLE 4 was placed into a compression die and tamped at less than 500 pounds per square inch (psi) to form a bottom layer. The core tablet was added.
(b) An additional amount of the blend from TABLE 4 was added on top of the tablet from step (a) and further compressed at 3000 lbs compression force in a Carver Press (Menomonee Falls, Wis.) equipped with 9 mm round, shallow concave tooling sets. The upper and lower layers for the compression coating portions were represented at a ratio of 1.29:1.
(c) The compression coated tablet from step (b) were tested for dissolution using a United States Pharmacopeia (USP) dissolution apparatus #1 (basket) at 100 RPM, in a dissolution media 0.1N HCl for 2 hours, followed by pH 5.6 phosphate buffer for 3-8 hours at 37 C.
(d) A set of tablets were prepared as in steps (a) and (b) at the same weight ratios (ratio of 1.29:1), with a total compression coating weight at 3× times the weight of the core, with 139 mg in the bottom portion and 179 mg in the upper portion, for a total target tablet weight of 424.38 mg. Three tablets were tested using the same dissolution method as in (c), resulting in a dissolution with less than 1 percent ibuprofen released before 6.25 hours (e.g., lag time of at least 4 hours), with an average dissolution rate of greater than 80% of ibuprofen released after 8 hours, at an average time of 403 minutes (6.71 hours). See FIG. 1.
(e) A set of tablets were prepared as in steps (a) and (b) at the same weight ratios (ratio of 1.29:1), with a total compression coating weight at 2.5 times the weight of the core, with 118 mg in the bottom portion and 152 mg in the upper portion, for a total target tablet weight of 376.38 mg. Three tablets were tested using the same dissolution method as in (c), resulting in a dissolution with less than 1 percent ibuprofen released before 4.5 hours (e.g., lag time of at least 4 hours), with an average dissolution rate of greater than 80% of ibuprofen released after 6 hours, at an average time of 310 minutes (5.17 hours). See FIG. 2.
(f) A set of tablets were prepared as in steps (a) and (b) at the same weight ratios (ratio of 1.29:1), with a total compression coating weight at 2.0 times the weight of the core, with 93 mg in the bottom portion and 119 mg in the upper portion, for a total target tablet weight of 318.38 mg. Three tablets were tested using the same dissolution method, resulting in a dissolution with less than 1 percent ibuprofen released before 3.5 hours (e.g., lag time of at least 3 hours), with an average dissolution rate of greater than 80% of ibuprofen released after 5 hours, at an average time of 238 minutes (3.96 hours). See FIG. 3.

TABLE 4
Pulse Release Compression Coating Formula

| Ingredient | % W/W | Manufacturer |
|---|---|---|
| Lactose (Fast Flow) | 55.00 | Foremost Farms |
| | | Baraboo, WI |
| Hydroxypropylmethylcellulose | 15.00 | The Dow Chemical |
| (HPMC K4M, CR Grade) | | Company, Midland, MI |
| Hydroxypropylcellulose | 30.00 | Aqualon |
| (HPC EXF) | | (Division of Hercules Inc.) |
| | | Wilmington, DE |
| Total (mg) | 100.0 | |

### Example 3

An additional 208.0 mg of the blend of ibuprofen, sodium starch glycolate (EXPLOTAB), and fumed silica (Cab-O-Sil) is added to the die containing the compression coated tablet to form an immediate release portion of the tablet. The tablet is additionally compressed to form a bilayer tablet.

### Example 4

### Dual Core Tablet: 200 mg Plus 100 mg

A dual core tablet is prepared utilizing the cores in Example 2. A 200 mg ibuprofen core is prepared by compressing 212.8 mg of the blend on a rotary press. 418.9 mg of the compression coating blend from Example 2 is placed into a round tablet die as a bottom layer, and the 200 mg core and 100 mg core from Example 2 are placed tangentially on top of the compression coating blend. An additional 538.6 mg of the compression coating blend is placed on top layer and the tablet is compressed. The 200 mg core is partially exposed on the surface of the tablet to provide immediate release, whereas one 100 mg core provides delayed release.

### Example 5

### Three Core Tablet: 100 mg Plus 100 mg Plus 100 mg

A three core tablet is prepared utilizing 100 mg core tablets from Example 2. 418.9 mg of the compression coating blend from Example 2 is placed into a round tablet die as a bottom layer, and three of the 100 mg core tablets from Example 2 are placed tangentially on top of the compression coating blend. An additional 538.5 mg of the compression coating blend is placed on top and the tablet is compressed. Two out of three 100 mg cores are partially exposed on two opposing side surfaces of the tablet to provide immediate release, whereas one core 100 mg tablet provides a delayed release in the center of the tablet.

### Example 6

### Dual Core Tablet: 150 mg Plus 150 mg

A dual core tablet is prepared utilizing the core in Example 2. 150 mg ibuprofen cores are prepared by compressing 159.6 mg of the blend on a rotary press per core. 418.9 mg of the compression coating blend from Example 2 is placed into a round tablet die as a bottom layer, and the two 150 mg cores are placed tangentially on top of the compression coating blend. An additional 538.5 mg of the compression coating blend is placed on top and the tablet is compressed. One of the two 150 mg cores is partially exposed on the surface of the tablet to provide immediate release, whereas the other 150 mg core provides delayed release.

### Example 7

### Three Core Tablet: 75 mg Plus 150 mg Plus 75 mg

A three core tablet is prepared utilizing cores in Example 2. 75 mg ibuprofen cores are prepared by compressing 79.8 mg of the blend on a rotary press per core. A 150 mg ibuprofen core is prepared by compressing 159.6 mg of the blend on a rotary press per core. 418.9 mg of the compression coating blend from Example 2 is placed into a round tablet die as a bottom layer, and the two 75 mg cores are placed tangentially on opposing sides of the 150 mg core on top of the compression coating blend. An additional 538.5 mg of the compression coating blend is placed on top and the tablet is compressed. The two 75 mg cores are partially exposed on opposite sides of the surface of the tablet to provide immediate release, whereas the 150 mg core is in the center and provides delayed release.

### Example 8

### Preparation of Compression Coated Tablet without Wax (Comparative Example)

The core that was used included ibuprofen sodium dihydrate, mesoporous silicon dioxide, and silicon dioxide.

### Blend in Table 5, Prepared According to the Following:

(g) The lactose, hydroxypropylmethylcellulose and hydroxypropylcellulose were de-lumped through a #40 mesh sieve.
(h) The delumped lactose, hydroxypropylmethylcellulose, hydroxypropylcellulose (a) were mixed in a suitable v-blender for 15 minutes at 25 RPM
(i) Silicon Dioxide was added and blended for an additional 3 minutes.

TABLE 5
COMPRESSION COATING BLEND

| Formula (tablet) | |
|---|---|
| Coating by weight of core | 2x |
| MATERIAL | % of Blend |
| Lactose monohydrate (Fast Flo 316) | 55.0% |
| Carnauba wax | — |
| Hydroxypropyl cellulose (Klucel EXF) | 30.0% |
| Hydroxypropyl methylcellulose (Methocel K4MCR) | 15.0% |
| Silicon dioxide (Cab-O-Sil) | — |
| Hydroxypropyl cellulose (Klucel HXF) | — |
| TOTAL | 100% |
| Delay between initial release and >90% release | 2.0 hours |

The coating in Table 5 was compressed using Method A below.

### Compression Coating—Method A and Method B:

Method A (for a 2.6× Coating Weight: 143 mg×2.6=371.8 mg)
1. Assembled the 5/16″ round flat faced beveled edge punch
2. Compressed core tablet (143 mg) at 0.5 tons of pressure
3. Assembled the 15/32″ round flat faced beveled edge punch
4. Placed 9/16^{th} (209.2 mg) of the coating material in die cavity
5. Gently placed the core tablet from Step #2 in the center of the punch on top of the coating material
6. Place the remaining 7/16^{th} (162.7 mg) of coating material on in the die cavity on top of the core tablet
7. Gently level material and compress at 0.75 tons
   Method B (for a 2.6× Coating Weight: 143 mg×2.6=371.8 mg
1. Assembled the 5/16″ round flat faced beveled edge punch
2. Lightly tamped 20% (74.4 mg) of the coating material
3. Added 143 mg of the core blend
4. Compressed the bi-layer tablet at 0.5 tons
5. Assembled the 15/32″ round FFBE punch
6. Centered the bi-layer tablet from Step #4 on the bottom punch and placed the remaining 80% (297.4 mg) of coating material on top of the bilayer tablet
7. Compressed the final tablet at 0.75 tons

### Example 9

### Compression Coating Blend

### Part A: Samples Using 10% Wax

The Blend from Table 6 was compressed at various weights using the core tablet from EXAMPLE 8, and the compression procedure below. The weights of the coating material were varied at 1.8×, 2×, 2.3× and 2.6× by weight of core tablet. In Trial 1 (Sample 1), silicon dioxide was added.

TABLE 6
Compression Coating Blend Formulations (as
a % of the formulation) 10% Carnuaba wax,
15% HPMC K4MCR, 30% HPC EXF, 45% lactose

| Formula (tablet) | Trial 1 | Trial 2 | Trial 3 | Trial 4 |
|---|---|---|---|---|
| Coating weight by weight | 1.8x | 2x | 2.3x | 2.6x |
| of core | | | | |
| MATERIAL | | | | |
| Lactose monohydrate | 44.5% | 45.0% | 45.0% | 45.0% |
| (Fast Flo 316)¹ | | | | |
| Carnauba wax | 10.0% | 10.0% | 10.0% | 10.0% |
| Hydroxypropyl cellulose, | 30.0% | 30.0% | 30.0% | 30.0% |
| HPC (Klucel EXF)² | | | | |
| Hydroxypropyl methyl- | 15.0% | 15.0% | 15.0% | 15.0% |
| cellulose, HPMC | | | | |
| (Methocel K4MCR)³ | | | | |
| Silicon dioxide (Cab-O-Sil) | 0.5% | — | — | — |
| Hydroxypropyl cellulose | — | — | — | — |
| (Klucel HXF)² | | | | |
| TOTAL | 100% | 100.0% | 100.0% | 100.0% |
| Delay until release (hours) | 2.0-3.0 | 3.0-3.5 | 3.5 | 6.0-6.5 |

¹Commerically available Foremost Farms Corporation in Baraboo, WI
²Commercially available from Ashland Corporation in Covington, KY
³Commercially available from the DOW Chemical Company in Midland, MI
4: In Table 6, the coatings were compressed using Method A (from Example 8) for Trial 4 and Method B (from Example 8) for Trials 1-3.

### Part B: Samples Using 30% Wax, 15/30% Ratio of HPMC K4MCR/HPC EXF

The Blend from Table 7 was compressed at various weights using the core tablet from EXAMPLE 8, and the compression procedure below. The weights of the coating material were compressed 2.6× by weight of core tablet. Multiple tablets were compressed in each sample variation.

TABLE 7
Compression Coating Blend Formulations
(as a % of the formulation), 30% Wax

| Formula (tablet) | Trial 5 |
|---|---|
| Coating weight by weight of core | 2.6x |
| MATERIAL | |
| Lactose monohydrate (Fast Flo 316) | 24.5% |
| Carnauba wax | 30.0% |
| Hydroxypropyl cellulose (Klucel EXF) | 30.0% |
| Hydroxypropyl methylcellulose (Methocel K4MCR) | 15.0% |
| Silicon dioxide (Cab-O-Sil) | 0.5% |
| Hydroxypropyl cellulose (Klucel HXF) | — |
| TOTAL | 100.0% |
| Delay (hours) | 6.0 |

In Table 7, the coating was compressed using Method B (from Example 8) for Trial 5.

### Part C: Samples Using 30% Wax, 13/26% Ratio of HPMC K4MCR/HPC EXF

The Blend from Table 8 was compressed at various weights using the core tablet from EXAMPLE 8, and the compression procedure below. The weights of the coating material were compressed 2.6× by weight of core tablet. Multiple tablets were compressed in each sample variation.

TABLE 8
Compression Coating Blend Formulations
(as a % of the formulation), 30% Wax

| Formula (tablet) | Trial 6 |
|---|---|
| Coating weight by weight of core | 2.6X |
| MATERIAL | |
| Lactose monohydrate (Fast Flo 316) | 30.0% |
| Carnauba wax | 30.0% |
| Hydroxypropyl cellulose (Klucel EXF) | 26.0% |
| Hydroxypropyl methylcellulose (Methocel K4MCR) | 13.0% |
| Silicon dioxide (Cab-O-Sil) | 1.0% |
| Hydroxypropyl cellulose (Klucel HXF) | |
| TOTAL | 100.0% |
| Delay (hours) | 4.5-5.67 |

In Table 8, the coating was compressed using Method B (from Example 8) for Trial 6.

### Part D: Samples Using 60% Wax

The Blend from Table 9 was compressed at various weights using the core tablet from EXAMPLE 8, and the compression procedure below. The weights of the coating material were compressed 2.6× by weight of core tablet. Multiple tablets were compressed in each sample variation.

TABLE 9
Compression Coating Blend Formulations (as a % of the formulation),
60% Wax 60% wax, 4% HXF, 35% lactose, 1% Cab-O-Sil

| Formula (tablet) | Trial 7 |
|---|---|
| Coating weight by weight of core | 2.6x |
| MATERIAL | |
| Lactose monohydrate (Fast Flo 316) | 35% |
| Carnauba wax | 60% |
| Hydroxypropyl cellulose (Klucel EXF) | — |
| Hydroxypropyl methylcellulose (Methocel K4MCR) | — |
| Silicon dioxide (Cab-O-Sil ® M-5) | 1% |
| Hydroxypropyl cellulose (Klucel HXF) | 4% |
| TOTAL | 100% |
| Delay (hours) | 4.5-5.0 |

In Table 9, the coating was compressed using Method B (from Example 8) for Trial 7.

### Example 10

### Preparation of Compression Coated Tablet with Wax

The tablet core that was used included phenylephrine HCL, mesoporous silica, and silicon dioxide for a core tablet weight of 155.0 mg and a dose of 138.8 mg of phenylephrine hydrochloride.

### Part a: Blend in Table 10, Prepared According to the Following:

(j) The lactose, hydroxypropylmethylcellulose and hydroxypropylcellulose were de-lumped through a #40 mesh sieve.
(k) The delumped lactose, hydroxypropylmethylcellulose, hydroxypropylcellulose (a) and carnauba wax were mixed in a suitable v-blender for 15 minutes at 25 RPM
(l) Silicon Dioxide was added and blended for an additional 3 minutes.

TABLE 10
COMPRESSION COATING BLEND

| Formula (tablet) | |
|---|---|
| Coating by weight of core | 2x |
| MATERIAL | % of Blend |
| Lactose monohydrate (Fast Flo 316) | 30.0% |
| Carnauba wax | 30.0% |
| Hydroxypropyl cellulose (Klucel EXF) | 26.0% |
| Hydroxypropyl methylcellulose (Methocel K4MCR) | 13.0% |
| Silicon dioxide (Cab-O-Sil) | 1.0% |
| TOTAL | 100% |
| Delay between initial release and >90% release | 2.0 hours |

In Table 10, the coating was compressed using Method B (from Example 8).

### Example 11

### Dissolution Results

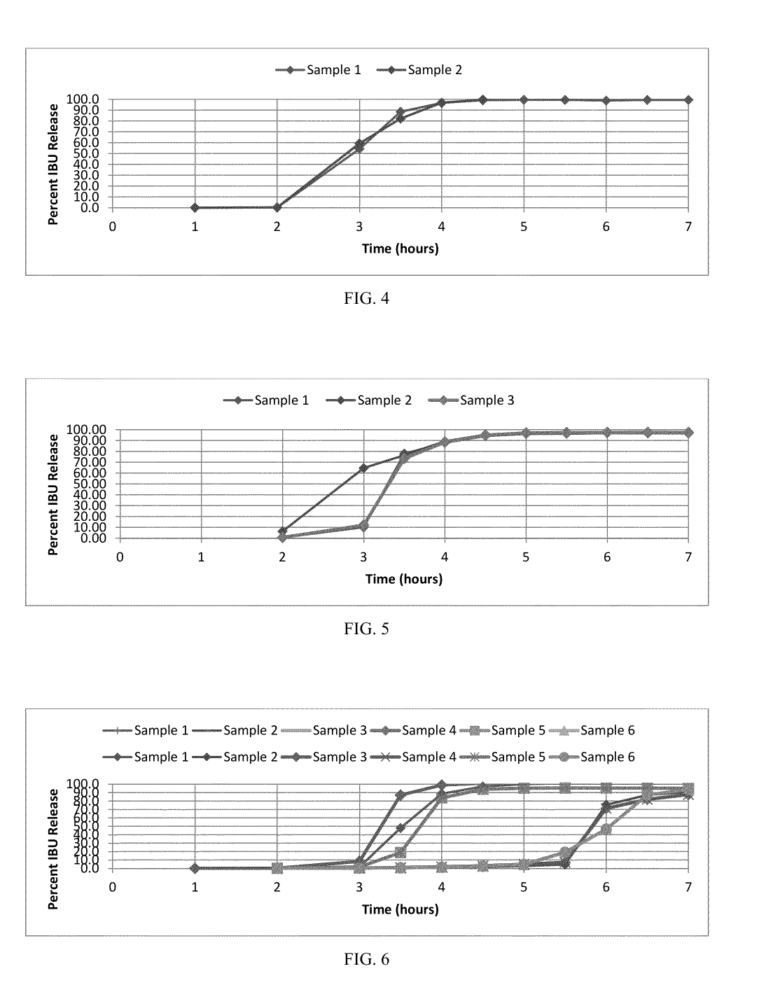

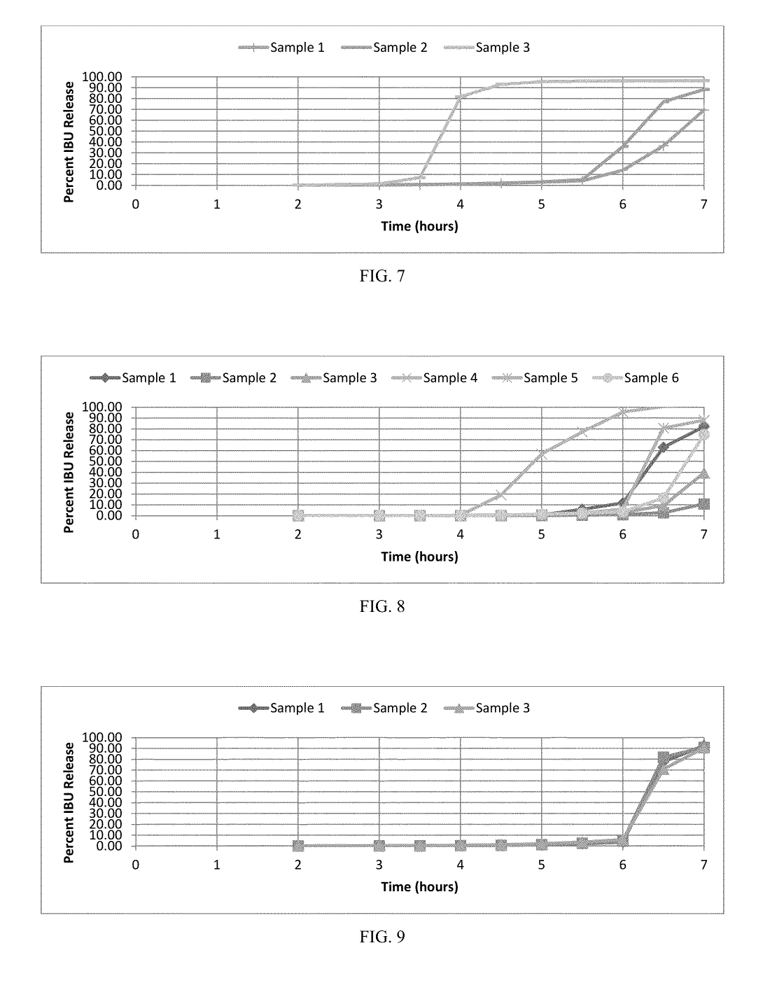

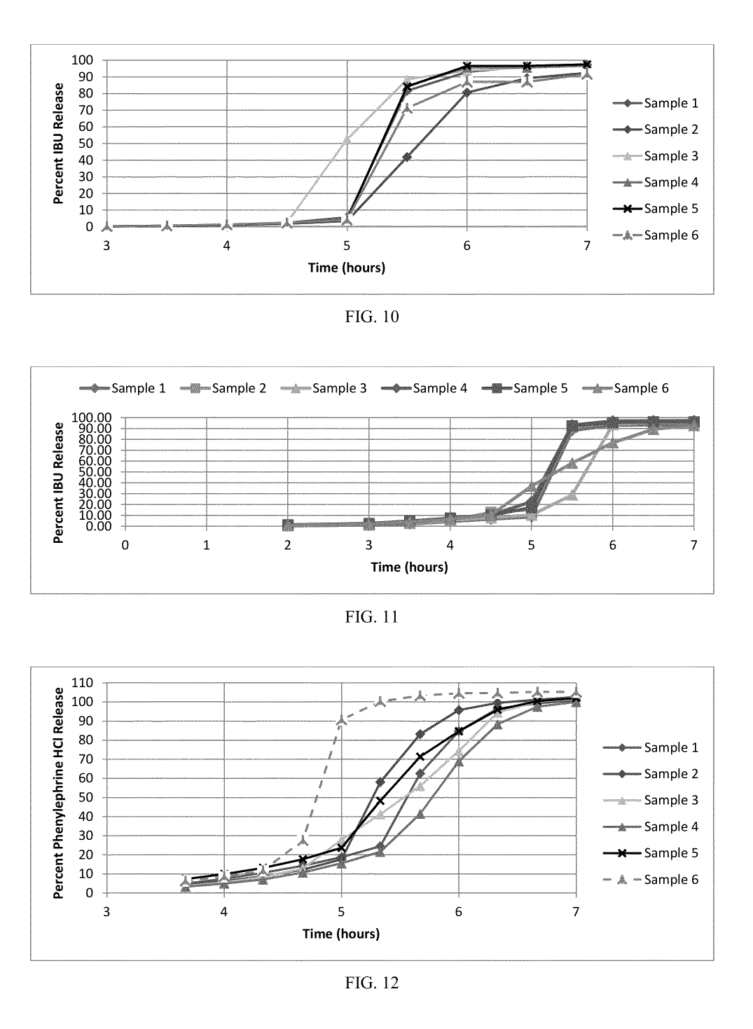

Tablets from Examples 8, 9 and 10 were tested for dissolution using a United States Pharmacopeia (USP) dissolution apparatus #1 (basket) at 100 RPM, in a dissolution media 0.1N HCL for 1 hour, followed by pH 6.8 phosphate buffer for 1-7 hours. Samples were tested for ibuprofen versus a standard at 100% released. The results include a degree of tablet to tablet variability.
(a) In the dissolution graph depicted in FIG. 4, tablets made using Compression Method A (from Example 8) displayed a lag time of ibuprofen release until 2 hours, and did not release greater than 90% of ibuprofen released until greater than 3.5 hours. This indicated that this formulation was not suitable for this active ingredient as a pulse release.
(b) In the dissolution graph depicted in FIG. 5, tablets from Example 9, Part A (10% wax) using Compression Method B (from Example 8) at a compression coating of 1.8× core weight did not release greater than 10% ibuprofen at 2 hours (indicating a lag time), and slowly released ibuprofen until 4 hours, indicating that a distinct pulse release was not displayed.
(c) In the dissolution graph depicted in FIG. 6, tablets from Example 9, Part A (10% Wax) using Compression Method A (from Example 8) at a compression coating of 2× core weight had a distinct pulse release at greater than 80% ibuprofen starting between 3 hours and 5.5 hours.
(d) Also, in the dissolution graph depicted in FIG. 6, tablets from Example 9, Part A (10% Wax) using Compression Method B (from Example 8) at a compression coating of 2× core weight had a distinct pulse release at greater than 80% ibuprofen starting between 3 hours and 5.5 hours.
(e) In the dissolution graph depicted in FIG. 7, tablets from Example 9, Part A (10% Wax) using Compression Method B (from Example 8) at a compression coating of 2.3× core weight displayed a varying release starting between 3.5 and 5.5 hours.
(f) In the dissolution graph depicted in FIG. 8, tablets from Example 9, Part A (10% Wax) using Compression Method A (from Example 8) at a compression coating of 2.6× core weight displayed a varying release starting between 4 and 6.5 hours, and did not display a distinct pulse release.
(g) In the dissolution graph depicted in FIG. 9, tablets from Example 9, Part B (30% Wax, 15% HPMC K4MCR, 30% HPC EXF) using Compression Method B (from Example 8) at a compression coating of 2.6× core weight displayed a delayed release rate of less than 10% ibuprofen released at 6 hours, followed by a distinct pulse with 90% released at 7 hours.
(h) In the dissolution graph depicted in FIG. 10, tablets from Example 9, Part C (30% Wax, 13% HPMC K4MCR, 26% HPC EXF) using Compression Method B (from Example 8) at a compression coating of 2.6× core weight displayed a delayed release rate with ibuprofen released starting between 4.5 and 5 hours, displaying a distinct pulse release with greater than 80% released at 6 hours.
(i) In the dissolution graph depicted in FIG. 11, tablets from Example 9, Part D (60% Wax) using Compression Method B (from Example 8) at a compression coating of 2.6× core weight displayed a release starting between 4 and 5 hours, with a greater than 80% released at 6 hours.
(j) In the dissolution graph depicted in FIG. 12, tablets from Example 10, (30% wax) at a compression coating of 2.6× core weight displayed a release starting between 4 and 5 hours, with a greater than 80% released at 6 hours.
   Additional tablets were prepared at the same weight ratios, with a total compression coating weight at 2.6 times the weight of the core, with 116 mg in the bottom portion and 150 mg in the upper portion, for a total target tablet weight of 372 mg. Three tablets were tested using the same dissolution method as in (c), resulting in a dissolution with less than 2 percent ibuprofen released after 4 hours (e.g., lag time of at least 4 hours), with an average dissolution rate of greater than 80% of ibuprofen released after 4 hours, at an average time of 310 minutes (5.17 hours).

### Example 12

### Dual Core Tablet: 200 mg Plus 100 mg

A dual core tablet is prepared utilizing the cores from Example 8. A 200 mg ibuprofen core is prepared by compressing 286 mg of the blend on a rotary press. 223.08 mg of the compression coating blend from Example 8, Part C (Table 5) is placed into a round tablet die as a bottom layer, and the 200 mg core and 100 mg core from Example 8 are placed tangentially on top of the compression coating blend. An additional 892.32 mg of the compression coating blend is placed on the top layer and the tablet is compressed. The 200 mg core is partially exposed on the surface of the tablet to provide immediate release, whereas one 100 mg core provides delayed release.

### Example 13

### Three Core Tablet: 100 mg Plus 100 mg Plus 100 mg

A three core tablet is prepared utilizing the 100 mg cores from Example 8. 223.08 mg of the compression coating blend from Example 8, Part C (Table 5) is placed into a round tablet die as a bottom layer, and three of the 100 mg core tablets from Example 8 are placed tangentially on top of the compression coating blend. An additional 892.32 mg of the compression coating blend is placed on top and the tablet is compressed. Two out of three 100 mg cores are partially exposed on two opposing side surfaces of the tablet to provide immediate release, whereas one core 100 mg tablet provides a delayed release in the center of the tablet.

### Example 14

### Dual Core Tablet: 150 mg Plus 150 mg

A dual core tablet is prepared utilizing the cores from Example 8. 150 mg ibuprofen cores are prepared by compressing 214.5 mg of the blend on a rotary press per core. 223.08 mg of the compression coating blend from Example 8 is placed into a round tablet die as a bottom layer, and the two 150 mg cores are placed tangentially on top of the compression coating blend. An additional 892.32 mg of the compression coating blend is placed on top and the tablet is compressed. One of the two 150 mg cores is partially exposed on the surface of the tablet to provide immediate release, whereas the other 150 mg core provides delayed release.

### Example 15

### Three Core Tablet: 75 mg Plus 150 mg Plus 75 mg

A three core tablet is prepared utilizing the cores from Example 8. 75 mg ibuprofen cores are prepared by compressing 107.25 mg of the blend on a rotary press per core. A 150 mg ibuprofen core is prepared by compressing 214.5 mg of the blend on a rotary press per core. 223.08 mg of the compression coating blend from Example 8 is placed into a round tablet die as a bottom layer, and the two 75 mg cores are placed tangentially on opposing sides of the 150 mg core on top of the compression coating blend. An additional 892.32 mg of the compression coating blend is placed on top and the tablet is compressed. The two 75 mg cores are partially exposed on opposite sides of the surface of the tablet to provide immediate release, whereas the 150 mg core is in the center and provides delayed release.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

What is claimed is:

1. A dosage form comprising an immediate release portion of a first active pharmaceutical ingredient and a delayed release portion of a second active pharmaceutical ingredient wherein (a) the immediate release portion comprises from about 1 mg to about 1000 mg of the first active pharmaceutical ingredient; and (b) the delayed release portion comprises from about 1 mg to about 1000 mg of the second active pharmaceutical ingredient; wherein the delayed release portion is coated with a delayed release coating comprising at least one swellable erodible polymer and a filler, and wherein the immediate release portion is in contact with the delayed release coating.

2. The dosage form of claim 1, wherein the swellable erodible polymer is selected from the group consisting of water swellable cellulose derivatives, polyalkalene glycols, thermoplastic polyalkalene oxides, acrylic polymers, hydrocolloids, gelling starches, and swelling cross-linked polymers, and derivatives, copolymers, and combinations thereof.

3. The dosage form of claim 1, wherein the swellable erodible polymer is selected from the group consisting of hydroxypropyl methylcellulose, hydroxypropylcellulose, hydroxyethylcellolose, and mixtures thereof.

4. The dosage form of claim 1, wherein the coating comprises two swellable erodible polymers.

5. The dosage form of claim 1, wherein the filler is selected from the group consisting of water insoluble polymers, lactose, dextrose, sucrose, mannose, mannitol, sorbitol, erythitol, xylitol, fats, fatty acid esters, phospholipids, waxes, vegetable oils, free fatty acids and their salts, phospholipids, and mixtures thereof.

6. The dosage form of claim 1, wherein the filler is selected from the group consisting of lactose, carnauba wax, and mixtures thereof.

7. The dosage form of claim 1, further comprising an optional ingredient selected from the group consisting of other actives, lubricants, glidants, sweeteners, colors, flavors, superdisintegrants, compressible fillers, and mixtures thereof.

8. The dosage form of claim 1, wherein the first active pharmaceutical ingredient within the immediate release portion and the second active pharmaceutical ingredient within the delayed release portion are the same.

9. The dosage form of claim 1, wherein the second active pharmaceutical ingredient is released about 4 to about 6 hours after the first active pharmaceutical ingredient.

10. The dosage form of claim 1, wherein the dosage form provides about 10-12 hours of treatment.

11. A dosage form comprising:
(a) an immediate release portion comprising from about 100 mg to about 400 mg of sodium ibuprofen;
(b) a delayed release portion comprising from about 50 mg to about 400 mg of sodium ibuprofen; and
(c) a coating surrounding the delayed release portion comprising at least one swellable erodible polymer and a filler; and
wherein the immediate release portion is in contact with the delayed release coating.

12. The dosage form of claim 11, wherein the swellable erodible polymer is selected from the group consisting of water swellable cellulose derivatives, polyalkalene glycols, thermoplastic polyalkalene oxides, acrylic polymers, hydrocolloids, clays, gelling starches, and swelling cross-linked polymers, and derivatives, copolymers, and combinations thereof.

13. The dosage form of claim 11, wherein the swellable erodible polymers are selected from the group consisting of hydroxypropyl methylcellulose, hydroxypropylcellulose, hydroxyethylcellolose, and mixtures thereof.

14. The dosage form of claim 11, wherein the filler is selected from the group consisting of water insoluble polymers, lactose, dextrose, sucrose, mannose, mannitol, sorbitol, erythitol, xylitol, fats, fatty acid esters, phospholipids, waxes, vegetable oils, free fatty acids and their salts, phospholipids, and mixtures thereof.

15. The dosage form of claim 11, wherein the filler is selected from the group consisting of lactose, carnauba wax, and mixtures thereof.

16. The dosage form of claim 11, wherein the delayed release portion is released about 4 to about 6 hours after the immediate release portion.

17. The dosage form of claim 11, wherein the sodium ibuprofen in the immediate release portion is about 200 mg, and the delayed release portion is 100 mg.

18. The dosage form of claim 11, wherein the sodium ibuprofen in the immediate release portion is about 150 mg, and the delayed release portion is 150 mg.

19. The dosage form of claim 11, wherein the ratio of the amount of ibuprofen sodium within the immediate release portion and within the delayed release portion is from about 2:1 to about 1:1.

20. The dosage form of claim 11, wherein the coating is comprised of at least two swellable erodible polymers.

21. A process for the manufacture of an immediate release portion of a first active pharmaceutical ingredient and a delayed release portion of a second active pharmaceutical ingredient, the method comprising:
(a) obtaining a core comprising from about 1 mg to about 1000 mg of a first active pharmaceutical ingredient;
(b) compressing a powder on the surface of the core to form a delayed release coating on the surface of the core, wherein the powder comprises at least one swellable erodible polymer; and
(c) compressing a second powder onto the surface of the delayed release coating, wherein the second powder comprises from about 1 mg to about 1000 mg of a first active pharmaceutical ingredient; wherein the immediate release portion comprises the compressed second powder, and the delayed release portion comprises the core and the delayed release coating.

22. A process of claim 21, wherein the core is formed by compressing a first powder comprising from about 50 mg to about 200 mg of ibuprofen sodium.

23. A process of claim 21, wherein the delayed release coating is formed by: adding a first portion of the powder to a die cavity; then adding the core to the die cavity containing the first portion of the powder; then adding a second portion of the powder to the die cavity; and then compressing the first portion of the powder, the core, and the second portion of the powder within the die cavity to form the delayed release coating on the surface of the core.

24. A process of claim 21, wherein the dosage form is formed by adding the second powder to a die cavity; then adding the core comprising the delayed release coating to the die cavity containing the second powder; and then compressing the second powder and the core comprising the delayed release coating within the die cavity to form the dosage form.

25. A process of claim 21, wherein the dosage form is formed by adding a first portion of the powder to a die cavity; then adding the core comprising the delayed release coating to the die cavity containing the first portion of the powder; then adding a second portion of the powder to the die cavity; and then compressing the first portion of the powder, the core comprising the delayed release coating, and the second portion of the powder within the die cavity to form the dosage form.

26. A process of claim 21, wherein the dosage is adapted both to release the ibuprofen within the immediate release portion within the first hour following administration of the dosage form and to release the ibuprofen within the delayed release portion from about 2 hours to about 8 following administration of the dosage form.

27. A process of claim 21, wherein the ibuprofen within the immediate release portion comprises ibuprofen sodium.

28. A process of claim 21, wherein the swellable erodible polymer is selected from the group consisting of hydroxypropyl methylcellulose, hydroxypropylcellulose, and hydroxyethylcellulose.

29. A process of claim 21, wherein the ratio of the amount of ibuprofen within the immediate release portion and within the delayed release portion is from about 2:1 to about 1:1.

30. A process of claim 21, wherein the immediate release portion comprises from about 50 to about 400 mg of ibuprofen.

31. A process of claim 21, wherein the delayed release portion comprises from about 50 to about 400 mg of ibuprofen.

32. A process of claim 21, wherein the powder comprises at least 15% by weight of a swellable erodible polymers.

33. A process of claim 21, wherein the powder comprises at least 15 percent by weight of a first swellable erodible polymer, and at least 15 percent by weight of a second swellable erodible polymer.

34. A dosage form manufactured by the process of claim 21.
